(19) United States
(12) Patent Application Publication
(10) Pub. No.: US 2025/0014686 A1
(43) Pub. Date: Jan. 9, 2025

(54) ESTIMATION APPARATUS AND ESTIMATION METHOD

(71) Applicant: Preferred Networks, Inc., Tokyo (JP)

(72) Inventors: Kohei SHINOHARA, Tokyo (JP); Kosuke NAKAGO, Tokyo (JP); Akihide HAYASHI, Tokyo (JP)

(73) Assignee: Preferred Networks, Inc., Tokyo (JP)

(21) Appl. No.: 18/896,110

(22) Filed: Sep. 25, 2024

Related U.S. Application Data
(63) Continuation of application No. PCT/JP2023/012321, filed on Mar. 27, 2023.

(30) Foreign Application Priority Data
Mar. 30, 2022 (JP) 2022-056104

(51) Int. Cl. G16C 20/30; G16C 20/70

(52) U.S. Cl. CPC G16C 20/30; G16C 20/70

(57) ABSTRACT

An estimation apparatus according to an embodiment includes at least one memory and at least one processor. At least one processor described above inputs a feature amount of each of a plurality of atoms to a neural network to update the feature amount, and generates a parameter corresponding to each of the plurality of atoms based on the updated feature amount. At least one processor described above determines each of a plurality of charges corresponding to each of the plurality of atoms by using the parameter.

### CROSS-REFERENCE TO RELATED APPLICATIONS

This application is based upon and claims the benefit of priority from Japanese Patent Application No. 2022-056104, filed on Mar. 30, 2022, and International Patent Application No. PCT/JP2023/012321 filed on Mar. 27, 2023; the entire contents of all of which are incorporated herein by reference.

### FIELD

An embodiment of the present invention relates to an estimation apparatus and an estimation method.

### BACKGROUND

There has been known a neural network that predicts overall energy of a state of an atom and force received by each atom (hereinafter, referred to as neural network potential (NNP)). The NNP can output energy and/or force in a time shorter than a time in simulation of an electronic state in a density functional theory (DFT) and the like.

In input to a conventional NNP, for example, the coordinates of an atom may be converted into a descriptor for satisfying various physically required symmetries. Therefore, the conventional NNP has difficulty in flexibly reflecting an environment around an atom of interest. Furthermore, when a graph neural network (GNN), which easily and flexibly reflects an environment around an atom of interest, is used as the NNP, convolution is executed only within a preset cutoff range in graph convolution in the GNN, so that charge transfer at a long range greatly exceeding the cutoff range may fail to be handled. Furthermore, increasing the number of convolution layers in the GNN for handling the charge transfer at a long range may greatly increase a calculation amount.

### SUMMARY OF THE INVENTION

An object of the invention is to provide an estimation apparatus capable of flexibly considering an environment around an atom and estimating handling of charge transfer.

An estimation apparatus according to an embodiment includes at least one memory and at least one processor. At least one processor described above inputs a feature amount of each of a plurality of atoms to a neural network to update the feature amount, and generates a parameter corresponding to each of the plurality of atoms based on the updated feature amount. At least one processor described above determines each of a plurality of charges corresponding to each of the plurality of atoms by using the parameter.

### BRIEF DESCRIPTION OF THE DRAWINGS

FIG. 1 is a block diagram illustrating an example of a hardware configuration of an estimation apparatus according to an embodiment;

FIG. 2 illustrates an example of functional blocks in a processor according to the embodiment;

FIG. 3 illustrates an example of a learned graph neural network (GNN) according to the embodiment;

FIG. 4 illustrates an example of processing at a stage subsequent to the learned graph neural network (GNN) according to the embodiment; and

FIG. 5 is a flowchart illustrating an example of a procedure of estimation processing according to the embodiment.

### DETAILED DESCRIPTION

An embodiment will be described in detail below with reference to the drawings.

### Embodiment

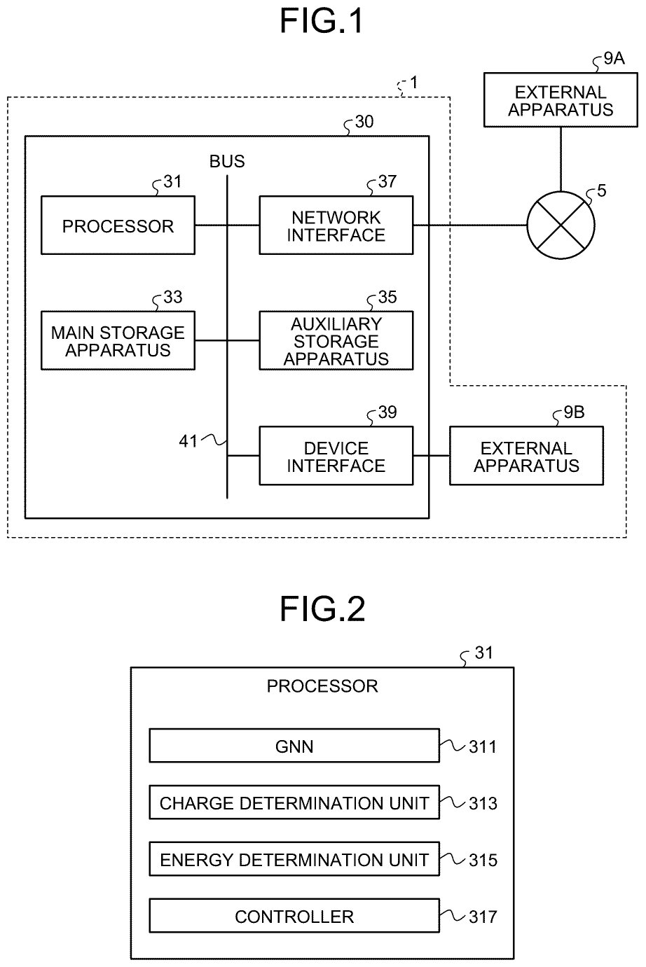

FIG. 1 is a block diagram illustrating an example of a hardware configuration of an estimation apparatus 1 according to the embodiment. As illustrated in FIG. 1, the estimation apparatus 1 may be connected to an external apparatus 9A via a communication network 5. Furthermore, the estimation apparatus 1 may include an external apparatus 9B connected via a device interface 39. The estimation apparatus 1 may estimate a plurality of charges corresponding to a plurality of atoms. The estimation apparatus 1 may estimate a plurality of pieces of energy corresponding to the plurality of atoms by using the plurality of estimated charges.

The estimation apparatus 1 includes a computer 30 and the external apparatus 9B connected to the computer 30 via the device interface 39. The computer 30 includes, for example, a processor 31, a main storage apparatus (memory) 33, an auxiliary storage apparatus (memory) 35, a network interface 37, and the device interface 39. The estimation apparatus 1 may be implemented as the computer 30 in which the processor 31, the main storage apparatus 33, the auxiliary storage apparatus 35, the network interface 37, and the device interface 39 are connected via a bus 41.

Although the computer 30 in FIG. 1 includes the components one by one, the computer 30 may have a plurality of the same components. Furthermore, although FIG. 1 illustrates one computer 30, software may be installed in a plurality of computers, and each of the plurality of computers may execute a part of the same or different processing of the software. In this case, a form of distributed computing may be adopted. In the distributed computing, computers communicate with each other via the network interface 37 and the like, and execute processing. That is, the estimation apparatus 1 in the embodiment may be configured as a system that implements various functions to be described later by one or a plurality of computers executing a command stored in one or a plurality of storage apparatuses. Furthermore, one or a plurality of computers provided on a cloud may process information transmitted from a terminal. The processing result may be transmitted to a terminal such as a display apparatus (display) corresponding to the external apparatus 9B.

Various arithmetic operations performed by the estimation apparatus 1 in the embodiment may be executed in parallel processing by using one or a plurality of processors or a plurality of computers via a network. Furthermore, various arithmetic operations may be distributed to a plurality of arithmetic cores in a processor, and executed in parallel processing.

Furthermore, a part or all of the processing, units, and the like of the present disclosure may be executed by at least one of a processor and a storage apparatus, provided on a cloud, capable of communicating with the computer 30 via the network. As described above, various types to be described later in the embodiment may be a form of parallel computing using one or a plurality of computers.

The processor 31 may be electronic circuitry (e.g., processing circuitry, central processing unit (CPU), graphics processing unit (GPU), field programmable gate array (FPGA), and application specific integrated circuitry (ASIC)) including a control apparatus and an arithmetic apparatus of the computer 30. Furthermore, the processor 31 may be a semiconductor device and the like including dedicated processing circuitry. The processor 31 is not limited to electronic circuitry using an electronic logic element, and may be implemented by optical circuitry using an optical logic element. Furthermore, the processor 31 may have an arithmetic function based on quantum computing.

The processor 31 can perform arithmetic processing based on data and software (program) input from each apparatus and the like of the internal configuration of the computer 30, and output the arithmetic result and a control signal to each apparatus and the like. The processor 31 may control each component constituting the computer 30 by executing an operating system (OS), an application, and the like of the computer 30.

The estimation apparatus 1 in the embodiment may be implemented by one or a plurality of processors 31. Here, the processor 31 may refer to one or a plurality of pieces of electronic circuitry disposed on one chip, or may refer to one or a plurality of pieces of electronic circuitry disposed on two or more chips or two or more devices. When a plurality of pieces of electronic circuitry is used, the plurality of pieces of electronic circuitry may communicate with each other by wire or wirelessly.

The main storage apparatus 33 stores a command, various pieces of data, and the like executed by the processor 31. The processor 31 reads information stored in the main storage apparatus 33. The auxiliary storage apparatus 35 is a storage apparatus other than the main storage apparatus 33. Note that these storage apparatuses mean any electronic component capable of storing electronic information, and may be semiconductor memories. The semiconductor memories may be either volatile memories or nonvolatile memories. A storage apparatus for storing various pieces of data used in an estimation apparatus 3 in the embodiment may be implemented by the main storage apparatus 33 or the auxiliary storage apparatus 35, or may be implemented by a built-in memory built in the processor 31. For example, a storage in the embodiment may be implemented by the main storage apparatus 33 or the auxiliary storage apparatus 35.

A plurality of processors may be connected (coupled) to one storage apparatus (memory). A single processor 31 may be connected to one storage apparatus (memory). A plurality of storage apparatuses (memories) may be connected (coupled) to one processor. When the estimation apparatus 1 in the embodiment includes at least one storage apparatus (memory) and a plurality of processors connected (coupled) to the at least one storage apparatus (memory), at least one of the plurality of processors may be connected (coupled) to the at least one storage apparatus (memory). Furthermore, the configuration may be implemented by a storage apparatus (memory) and the processor 31 included in a plurality of computers. Moreover, a storage apparatus (memory) may be integrated with the processor 31 (e.g., cache memory including L1 cache and L2 cache).

The network interface 37 is used for connection with the communication network 5 in a wireless or wired manner. An appropriate interface such as those adapted to an existing communication standard is required to be used as the network interface 37. Exchange of information with the external apparatus 9A connected via the communication network 5 may be performed by the network interface 37. Note that the communication network 5 may be any of a wide area network (WAN), a local area network (LAN), a personal area network (PAN), or the like, or a combination thereof. Exchange of information between the computer 30 and the external apparatus 9A is only required to be performed in the communication network 5. Examples of the WAN include the Internet. Examples of the LAN include IEEE802.11 and Ethernet (registered trademark). Examples of the PAN include Bluetooth (registered trademark) and near field communication (NFC).

The device interface 39 is an interface, such as a universal serial bus (USB), directly connected to an output apparatus such as a display apparatus, an input apparatus, and the external apparatus 9B. Note that, the output apparatus may include a speaker and the like that output voice and the like.

The external apparatus 9A is connected to the computer 30 via a network. The external apparatus 9B is directly connected to the computer 30.

In an example, the external apparatus 9A or 9B may be an input apparatus (input unit). Examples of the input apparatus include a camera, a microphone, a motion capture, various sensors, a keyboard, a mouse, and a touch panel. The input apparatus gives acquired information to the computer 30. Furthermore, the external apparatus 9A or 9B may be, for example, a personal computer, a tablet terminal, and a device including an input unit, a memory, and a processor, such as a smartphone.

Furthermore, the external apparatus 9A or 9B may be an output apparatus (output unit) in an example. The output apparatus may be, for example, a display apparatus (display) such as a liquid crystal display (LCD), a cathode ray tube (CRT), a plasma display panel (PDP), and an organic electro luminescence (EL) panel, or may be, for example, a speaker that outputs voice and the like. Furthermore, the external apparatus 9A or 9B may be, for example, a personal computer, a tablet terminal, and a device including an output unit, a memory, and a processor, such as a smartphone.

Furthermore, the external apparatus 9A or 9B may be a storage apparatus (memory). For example, the external apparatus 9A may be a network storage or the like. The external apparatus 9B may be a storage such as an HDD.

Furthermore, the external apparatus 9A or 9B may have a part of functions of components of the estimation apparatus 1 in the embodiment. That is, the computer 30 may transmit or receive a part or all of a processing result from the external apparatus 9A or 9B.

FIG. 2 illustrates an example of functional blocks in the processor 31. The processor 31 includes, for example, a graph neural network (GNN) 311, a charge determination unit 313, an energy determination unit 315, and a controller 317 as functions implemented by the processor 31. For example, the main storage apparatus 33 or the auxiliary storage apparatus 35 stores functions implemented by the GNN 311, the charge determination unit 313, the energy determination unit 315, and the controller 317 as programs. The processor 31 can implement the functions related to the GNN 311, the charge determination unit 313, the energy determination unit 315, and the controller 317 by reading and executing a program stored in the main storage apparatus 33 or the auxiliary storage apparatus 35.

The GNN 311 is, for example, a learned graph neural network including an input layer, a plurality of graph convolution layers, and an output layer. Learning for the GNN before learning will be described later. For example, settings (hereinafter, referred to as input information) related to a plurality of atoms to be estimated are input to the input layer. The input information includes the positional relation (coordinates) between a plurality of atoms in a substance, a structural relation of the plurality of atoms (e.g., structural expression of compound including plurality of atoms), the atomic number of each of the plurality of atoms, and the total charges of the plurality of atoms. The input layer may generate a graph indicating the relation between the plurality of atoms based on the input information. The graph may include nodes (also referred to as vertices) corresponding to the plurality of atoms and sides (also referred to as links) indicating a structural relation between the plurality of atoms and connecting the nodes. The graph is expressed as a matrix, for example. Furthermore, the input layer may determine feature amounts allocated to the plurality of nodes corresponding to the plurality of atoms based on the input information and a correspondence table for converting an atom into a vector. In such a manner, the input layer may represent each of the plurality of atoms by a feature amount, and input a structure of a substance including the plurality of atoms to a graph convolution layer at the first stage as a graph.

A plurality of graph convolution layers may maintain the input graph, and repeat graph convolution of a feature amount within a preset cutoff range (also referred to as cutoff radius). In the plurality of graph convolution layers, a range within which convolution is preliminarily performed for the graph may be preset as the cutoff radius. Note that the cutoff radius can be appropriately set by an instruction from a user via the input apparatus. A graph convolution layer at the last stage may output vectors indicating feature amounts corresponding to the plurality of atoms.

The output layer may output a plurality of parameters corresponding to the plurality of atoms based on the feature amounts calculated by the graph convolution layer at the last stage. Hereinafter, for the sake of concrete descriptions, it is assumed that each of the plurality of parameters has, in each of the plurality of atoms, an index indicating a degree of easiness of being electrically biased (hereinafter, referred to as electronegativity index) and an index indicating a degree of hardness of charge variation (hereinafter, referred to as hardness). The electronegativity index corresponds to atomic electronegativity. The above-described hardness corresponds to hardness in Density functional theory. Note that an index included in each of the parameters is not limited to the above-described two types, and can be appropriately set in accordance with learning of the GNN 311 and the like. For example, in each of the plurality of atoms, each of the plurality of parameters corresponds to at least one of the electronegativity index and the hardness, that is, the electronegativity index and/or the hardness. Details of the GNN 311 are based on known ones, and thus detailed description thereof will be omitted.

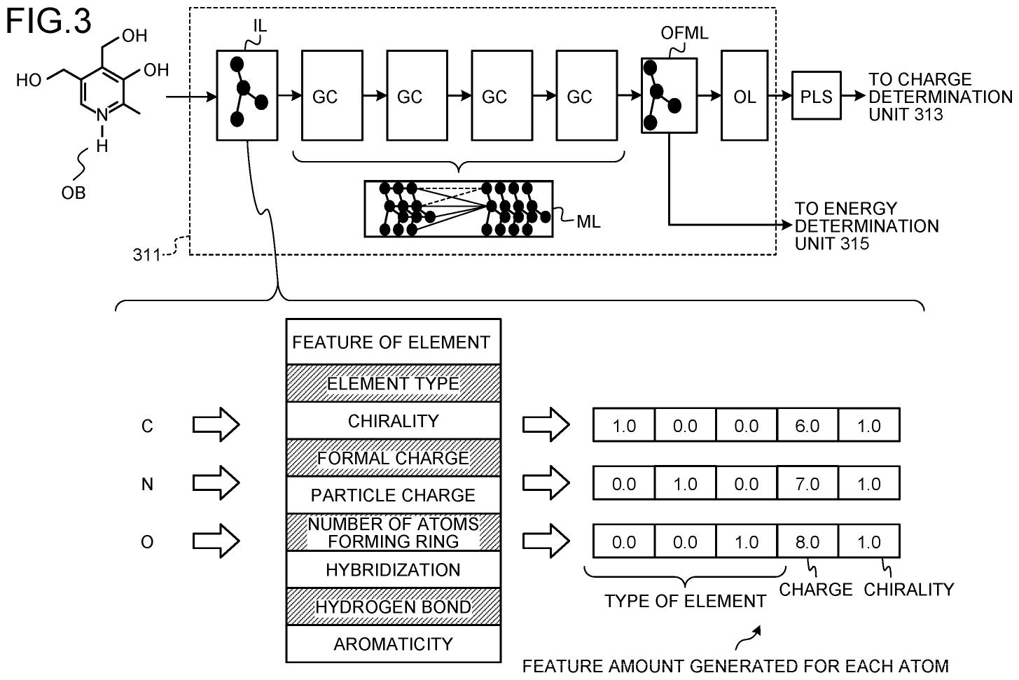

FIG. 3 illustrates an example of the learned GNN 311. A plurality of atoms OB to be estimated may be input to an input layer IL. As illustrated in FIG. 3, the input layer IL may generate a feature amount for each of atoms, such as elements C (carbon), N (nitrogen), and O (oxygen) in the plurality of atoms OB. The generated feature amount may be attached to a node. As illustrated in FIG. 3, a plurality of convolution layers GC may form a middle layer ML. Each of the plurality of convolution layers may execute graph convolution on feature amounts of a plurality of atoms (nodes) included in the cutoff range. Although FIG. 3 illustrates four (hereinafter, referred to as convolution number) graph convolution layers, this is not a limitation. Any number of graph convolution layers can be set.

As illustrated in FIG. 3, vectors OFML indicating feature amounts corresponding to the plurality of atoms output from the graph convolution layer at the last stage may be output to an output layer OL and the energy determination unit 315. The output layer OL may generate a plurality of parameters PLS corresponding to the plurality of atoms by performing linear transformation (e.g., matrix multiplication) on the vectors having the feature amounts of the plurality of atoms. For example, when a vector of a feature amount for one atom is a vector of the feature amount element number n×1, a value of a parameter is output by multiplying a matrix of the parameter element number (e.g., two components of electronegativity index and hardness)×n from the left side of the vector of the feature amount of n×1. As illustrated in FIG. 3, the output layer OL may output the plurality of parameters PLS corresponding to the plurality of atoms to the charge determination unit 313.

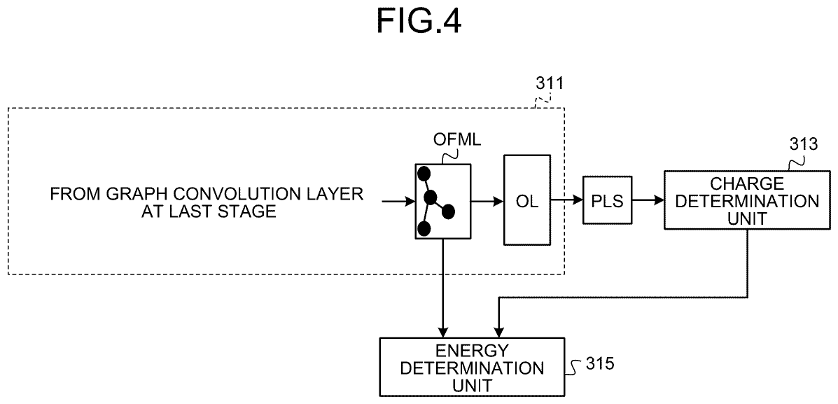

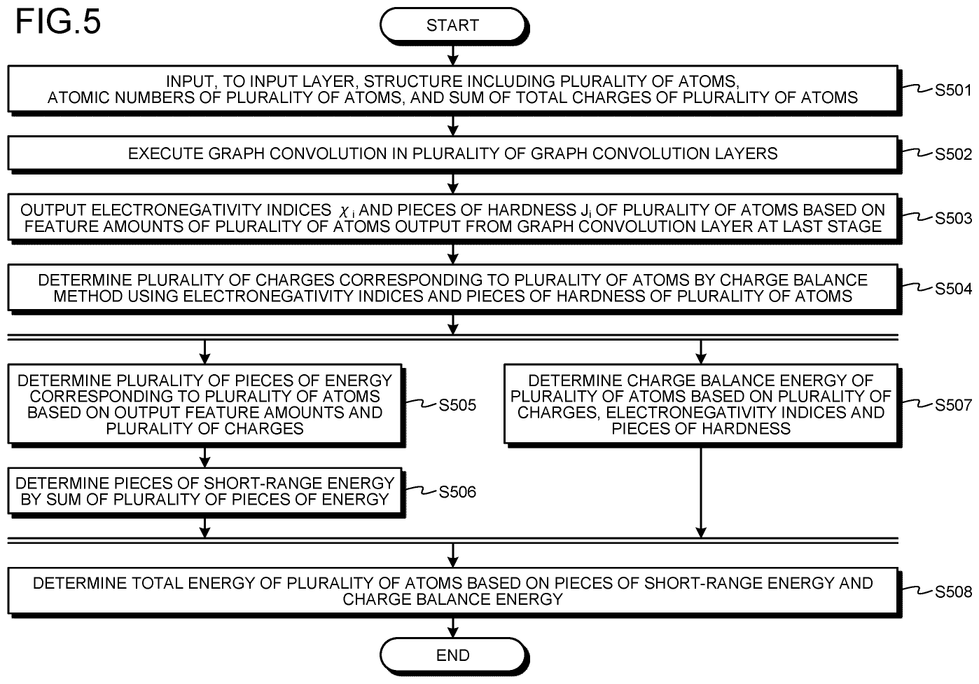

FIG. 4 illustrates an example of processing at a stage subsequent to the GNN 311. As illustrated in FIG. 4, the plurality of parameters PLS may be input to the charge determination unit 313. Furthermore, the vectors OFML indicating the feature amounts corresponding to the plurality of atoms and a plurality of charges corresponding to the plurality of atoms determined by the charge determination unit 313 may be output to the energy determination unit 315.

The charge determination unit 313 may determine the plurality of charges corresponding to the plurality of atoms by a charge balance method (also referred to as charge equilibration (Qeq) scheme) using the plurality of parameters PLS output from the output layer. The charge balance method is an approach of calculating charge balance distribution in a plurality of atoms to be estimated. Specifically, the charge determination unit 313 may determine the plurality of charges corresponding to the plurality of atoms by minimizing energy of charge balance (hereinafter, referred to as charge balance energy) such that the sum of the plurality of charges corresponding to the plurality of atoms is the total charges. The charge determination unit 313 uses Expression (1) below, for example.

$${min}_{\{ {Q}_{j} \} s . t . {\sum }_{i} {Q}_{i} = {Q}_{tot}} {E}_{Qeq} \qquad ( 1 )$$

E_{Qeq} in Expression (1) represents the charge balance energy. Qₜₒₜ in Expression (1) represents the total charges. Qⱼ in Expression (1) represents the charge of the jth atom in the plurality of atoms. Σ_{1Q1} in Expression (1) represents the sum of all the charges of the plurality of atoms. The charge balance energy E_{Qeq} is represented in Expression (2) below by using electrostatic energy Eₑₗₑ by a charge balance method of predicting an atomic charge represented by a Gaussian charge density of a width σ₁ indicated by a covalent bond radius of each element.

$${E}_{Qeq} = {E}_{ele} + \text{?} ( {χ}_{i} {Q}_{i} + \frac{1}{2} {J}_{i} {Q}_{i}^{2} ) \qquad ( 2 )$$

$$\text{?} \text{indicates text missing or illegible when filed}$$

Here, x₁ on the right side in Expression (2) represents the electronegativity index of the ith atom. Furthermore, J₁ on the right side in Expression (2) represents the hardness of the ith atom. Furthermore, Nₐₜ represents the total number of the plurality of atoms to be estimated. Furthermore, the electrostatic energy Eₑₗₑ in Expression (2) is represented by Expression (3) below including the Coulomb potential.

$${E}_{ele} = \text{?} \frac{erf ( \frac{{r}_{ij}}{\sqrt{2} {γ}_{ij}} )}{{r}_{ij}} \text{?} + \text{?} \frac{{Q}_{i}^{2}}{2 \text{?} \sqrt{π}} \qquad ( 3 )$$

$$\text{?} \text{indicates text missing or illegible when filed}$$

Here, γᵢⱼ in Expression (3) is defined in Expression (4) below by using a width σ₁ from the covalent bond radius of each element. Since Expression (3) is known, the description thereof will be omitted.

$${γ}_{ij} = \sqrt{{σ}_{i}^{2} + {σ}_{j}^{2}} \qquad ( 4 )$$

As illustrated in Expression (1), the charge determination unit 313 may determine the plurality of charges corresponding to the plurality of atoms so as to minimize the charge balance energy E_{Qeq} under a constraint condition that the sum of the plurality of charges corresponding to the plurality of atoms is the total charges (Σ₁Q₁=Qₜₒₜ). Expression (5) below gives an expression for determining the minimum of the charge balance energy E_{Qeq} by using Expressions (2) to (4) based on a relation of the partial differential of a charge to the charge balance energy E_{Qeq} being equal to zero.

$$\frac{\partial {E}_{Qeq}}{\partial {Q}_{i}} = 0 , \qquad ( 5 )$$

$$∀ i = 1 , … , {N}_{at} ⇒ {\sum }_{j = 1}^{{N}_{at}} {A}_{ij} {Q}_{j} + {χ}_{i} = 0$$

Expression (6) below defines a matrix Aᵢⱼ in Expression (5).

$${[ A ]}_{ij} = \{ \begin{matrix}{J}_{i} + \frac{1}{{σ}_{i} \sqrt{π}} , & if i = j \\ \frac{erf ( \frac{{r}_{ij}}{\sqrt{2} {γ}_{ij}} )}{\text{?}} , & otherwise\end{matrix} \qquad ( 6 )$$

$$\text{?} \text{indicates text missing or illegible when filed}$$

Expression (7) below is obtained by adding the constraint condition that the sum of the plurality of charges corresponding to the plurality of atoms is the total charges (Σ₁Q₁=Qₜₒₜ) to Expression (5).

$$( \begin{matrix} & & & 1 \\ & A & & ⋮ \\ & & & 1 \\ 1 & ⋮ & 1 & 0\end{matrix} ) ( \begin{matrix}{Q}_{1} \\ ⋮ \\ \text{?} \\ λ\end{matrix} ) = ( \begin{matrix}- {χ}_{1} \\ ⋮ \\ \text{?} \\ {Q}_{tot}\end{matrix} ) \qquad ( 7 )$$

$$\text{?} \text{indicates text missing or illegible when filed}$$

The charge determination unit 313 may determine the plurality of charges (Q₁, . . . , and Q_{Nat}) corresponding to the plurality of atoms by solving the equation in Expression (7). Specifically, the charge determination unit 313 may determine the plurality of charges (Q₁, . . . , and Q_{Nat}) by calculating an inverse matrix of the left side of Expression (7) and multiplying both the sides of Expression (7) by the calculated inverse matrix. Solving the inverse matrix corresponds to handling remote interaction between the plurality of charges in the plurality of atoms, that is, charge transfer (picture of charge transfer) at a long range. That is, even if the influence range of a feature amount is multiplied by the convolution number of the cutoff range due to a plurality of graph convolutions performed by the plurality of graph convolution layers GC, the charge determination unit 313 can determine the plurality of charges (Q₁, . . . , and Q_{Nat}) in consideration of charge transfer at a long range within the entire range over the plurality of atoms. The charge determination unit 313 can use a function for solving the above-described inverse matrix as an algorithm in a differentiable form. Therefore, backpropagation in learning of the GNN can also be applied to an inverse matrix.

The energy determination unit 315 may determine the plurality of pieces of energy corresponding to the plurality of atoms based on the plurality of charges and the feature amount calculated by the graph convolution layer at the last stage. Specifically, the energy determination unit 315 adds, in each of the plurality of atoms, the charge determined by the charge determination unit 313 to the feature amount calculated by the graph convolution layer at the last stage (concat).

Specifically, the energy determination unit 315 generates, in each of the plurality of atoms, a vector obtained by concatting a charge to a vector having a feature amount (hereinafter, referred to as feature amount charge vector). Next, the energy determination unit 315 may determine, in each of the plurality of atoms, energy of each of the plurality of atoms by performing linear transformation (e.g., matrix multiplication) on the feature amount charge vector. For example, when a vector of a feature amount for one atom is a vector of the feature amount element number n×1, the energy determination unit 315 generates a feature amount charge vector of (n+1)×1 by adding the charge of the atom. Next, the energy determination unit 315 determines the energy of the atom by multiplying a matrix of 1× (n+1) from the left side of the feature amount charge vector of (n+1)×1. The energy determination unit 315 may determine, in each of the plurality of atoms, the energy of each of the plurality of atoms by passing a neural network including nonlinear transformation to the feature amount charge vector.

The energy determination unit 315 may determine charge balance energy from the plurality of atoms based on the plurality of charges and the output parameters, and determine total energy over the plurality of atoms based on the plurality of pieces of energy corresponding to the plurality of atoms and the charge balance energy. Specifically, the energy determination unit 315 may calculate the electrostatic energy Eₑₗₑ by applying the plurality of charges (Q₁, . . . , and Q_{Nat}) and the positional relation (coordinates, width indicated by covalent bond radius of each element) between the plurality of atoms to Expression (3). Next, the energy determination unit 315 may calculate the second term on the right side of Expression (2) by using the plurality of charges (Q₁, . . . , and Q_{Nat}), the electronegativity index x₁, and hardness J₁. Finally, the energy determination unit 315 may determine the charge balance energy E_{Qeq} by Expression (2) by adding the electrostatic energy Eₑₗₑ and the calculated second term of the right side of Expression (2).

Furthermore, the energy determination unit 315 may calculate short-range energy by adding the plurality of pieces of energy corresponding to the plurality of atoms. Finally, the energy determination unit 315 may determine the total energy over the plurality of atoms by adding the short-range energy and the charge balance energy E_{Qeq}.

The controller 317 may control various components in the estimation apparatus 3. For example, the controller 317 may control functions related to the GNN 311, the charge determination unit 313, and the energy determination unit 315. Furthermore, the controller 317 may store at least one of the determined charges and various types of energy in the memory. Furthermore, the controller 317 may control various operations in the estimation apparatus 3.

The configuration of the estimation apparatus 3 has been described above. Processing of the estimation apparatus 3 estimating a plurality of charges and various types of energy (hereinafter, referred to as estimation processing) will be described below.

FIG. 5 is a flowchart illustrating an example of a procedure of the estimation processing.

### (Estimation Processing)

### (Step S501)

The input apparatus may input input information on a plurality of atoms to be estimated to the estimation apparatus 3 through an operation from the user. The controller 317 may input the input information to the GNN 311. Specifically, the controller 317 may input, to the input layer IL, a structure of a substance including a plurality of atoms, the atomic numbers of the plurality of atoms, and the sum of the total charges of the plurality of atoms. The input layer IL may input a graph to which feature amounts representing the plurality of atoms are attached to the graph convolution layer at the first stage.

### (Step S502)

The middle layer ML consisting of the plurality of graph convolution layers GC may execute graph convolution on the feature amounts related to the plurality of atoms included in the cutoff range in accordance with the graph and the cutoff range. The graph convolution layer at the last stage may output, to the output layer OL, the feature amounts of the plurality of atoms output from the graph convolution layer at the last stage. Furthermore, the graph convolution layer at the last stage may output, to the energy determination unit 315, the feature amounts of the plurality of atoms output from the graph convolution layer at the last stage.

### (Step S503)

The output layer OL may output electronegativity indices x₁ and pieces of hardness J₁ of the plurality of atoms based on feature amounts of the plurality of atoms output from the graph convolution layer at the last stage. The output layer OL may output the electronegativity indices x₁ and the pieces of hardness J₁ of the plurality of atoms to the charge determination unit 313. Note that the GNN 311 may store the plurality of output electronegativity indices and the plurality of pieces of output hardness in the memory in association with the plurality of atoms. Furthermore, the charge determination unit 313 may cause the display apparatus to display the plurality of electronegativity indices and the plurality of pieces of hardness in association with the plurality of atoms.

### (Step S504)

The charge determination unit 313 may determine the plurality of charges corresponding to the plurality of atoms by the charge balance method using the electronegativity indices and the pieces of hardness of the plurality of atoms. The charge determination unit 313 may output the plurality of determined charges to the energy determination unit 315. Note that the charge determination unit 313 may store values of the plurality of charges in the memory in association with the plurality of atoms. Furthermore, the charge determination unit 313 may cause the display apparatus to display the values of the plurality of charges in association with the plurality of atoms.

### (Step S505)

The energy determination unit 315 may determine the plurality of pieces of energy corresponding to the plurality of atoms based on the output feature amounts and the plurality of charges. The energy determination unit 315 may store values of the plurality of pieces of energy in the memory in association with the plurality of atoms. Furthermore, the energy determination unit 315 may cause the display apparatus to display the values of the plurality of pieces of energy in association with the plurality of atoms.

### (Step S506)

The energy determination unit 315 may determine pieces of short-range energy of the plurality of atoms by calculating the sum of the plurality of pieces of energy. The energy determination unit 315 may store values of the pieces of short-range energy in the memory in association with an estimation target. Furthermore, the energy determination unit 315 may cause the display apparatus to display the values of the pieces of short-range energy in association with the estimation target.

### (Step S507)

The energy determination unit 315 may determine the charge balance energy E_{Qeq} of the plurality of atoms based on the plurality of charges, the electronegativity indices, and the pieces of hardness. Specifically, the charge balance energy E_{Qeq} may be determined by applying the plurality of charges (Q₁, . . . , and Q_{Nat}), the positional relation between the plurality of atoms, the electronegativity indices x₁, and the pieces of hardness J₁ to Expressions (2) to (4). The energy determination unit 315 may store the value of the charge balance energy E_{Qeq} in the memory in association with the estimation target. Furthermore, the energy determination unit 315 may cause the display apparatus to display the value of the charge balance energy E_{Qeq} in association with the estimation target.

### (Step S508)

The energy determination unit 315 may determine the total energy of the plurality of atoms based on the pieces of short-range energy and the charge balance energy E_{Qeq}. The energy determination unit 315 may store the value of the total energy in the memory in association with the estimation target. Furthermore, the energy determination unit 315 may cause the display apparatus to display the value of the total energy in association with the estimation target.

In an application example of the embodiment, when a plurality of atoms to be estimated has a periodic boundary condition, the energy determination unit 315 can calculate various types of energy (e.g., electrostatic energy Eₑₗₑ and electrostatic interaction energy) by Ewald summation using the plurality of determined charges.

Furthermore, in a further application example of the embodiment, the energy determination unit 315 may calculate Van der Waals forces by using the plurality of determined charges as inputs to potential that needs charge input, such as density functional theory (DFT) dispersion (D) 4, chemistry at harvard macromolcular mechanics (CHARMM), and assisted model building with energy refinement (AMBER). In another application example of the embodiment, learning may be performed so that the same elements in the plurality of atoms to be estimated output common parameters.

Furthermore, in a further application example, learning may be performed so that even the same elements have different values of the width of for each of the plurality of atoms to be estimated, or so that the same elements in the plurality of atoms to be estimated have common values.

The estimation apparatus 3 according to the embodiment represents a plurality of atoms in feature amounts, and inputs the structure of a substance including the plurality of atoms as a graph. The estimation apparatus 3 calculates the updated feature amounts by maintaining the graph and repeating graph convolution of the feature amounts within a preset cutoff range. The estimation apparatus 3 outputs a plurality of parameters corresponding to the plurality of atoms based on the calculated feature amounts. The estimation apparatus 3 thus can determine a plurality of charges corresponding to the plurality of atoms by the charge balance method using the plurality of parameters. In the estimation apparatus 3 according to the embodiment, each of the plurality of parameters has, in each of the plurality of atoms, an index (electronegativity index) indicating a degree of easiness of being electrically biased and an index (hardness) indicating a degree of hardness of charge variation, for example.

Therefore, the estimation apparatus 3 according to the embodiment can output parameters (e.g., electronegativity indices and pieces of hardness) related to transfer of the plurality of charges corresponding to the plurality of atoms by using the learned GNN 311, and accurately determine the plurality of charges by the charge balance method using the parameters. Therefore, the estimation apparatus 3 according to the embodiment enables flexible consideration of an environment around the atoms by using the GNN 311. Moreover, the estimation apparatus 3 can handle charge transfer at a long range without increasing the number of convolution layers in the GNN, that is, without greatly increasing a calculation amount by using the charge balance method.

The estimation apparatus 3 according to the embodiment determines the plurality of pieces of energy corresponding to the plurality of atoms based on the plurality of charges and the calculated feature amounts. Furthermore, the estimation apparatus 3 according to the embodiment determines charge balance energy from the plurality of atoms based on the plurality of charges and the parameters, and determines total energy over the plurality of atoms based on the plurality of pieces of energy and the charge balance energy. As a result, the estimation apparatus 3 according to the embodiment can determine various types of energy related to the plurality of atoms by the charge transfer at a long range in flexible consideration of an environment around the atoms.

As a result, the estimation apparatus 3 according to the embodiment can estimate parameters necessary for the charge balance method in more flexible consideration of a surrounding environment by using the learned GNN 311. The estimation apparatus 3 can reduce a calculation cost by using the charge balance method, and determine the plurality of charges in consideration of long-range transfer of the charges. Therefore, the estimation apparatus 3 according to the embodiment can determine various types of energy in flexible consideration of a surrounding environment and the charge transfer at a long range.

Processing of causing the GNN before learning to be learned will be described below in relation to the GNN 311 according to the embodiment. A learning apparatus (also referred to as training apparatus) executes learning for the GNN before learning by using teacher data (also referred to as correct answer data) and training data input to the GNN to be learned in accordance with the teacher data. The learning apparatus may be implemented by, for example, components in the frame of 30 in FIG. 1. In the case, the processor in the learning apparatus includes a learning unit.

The teacher data and the training data used for learning correspond to learning data. Since a known approach such as the backpropagation can be applied to learning for the GNN before learning using the teacher data and the training data, description thereof will be omitted.

The training data corresponds to input information input to the GNN before learning. For example, a result obtained by calculation (quantum simulation) through Density functional theory (DFT) by using the input information is used as the teacher data. In the case, the teacher data includes information on the total energy of the plurality of atoms and information on the plurality of charges of the plurality of atoms. The information on the charges relates to, for example, Hirshfeld charges. In the case, the DFT calculates Hirshfeld charges based on the input information. The charge determination unit 313 determines Hirshfeld charges.

Note that the information on the charges is not limited to Hirshfeld charges, and may relate to point charges such as Bader charges and Mulliken charges. In the case, the DFT calculates Bader charges or Mulliken charges based on the input information. The charge determination unit 313 determines Bader charges or Mulliken charges. Hirshfeld charges, Bader charges, and Mulliken charges correspond to charges in spatial distribution of the probability of electrons calculated by Schrodinger equation.

Furthermore, not only the point charges but dipole moments of charges may be used as the teacher data. The GNN before learning may be learned such that Σrᵢ*qᵢ satisfies dipole=∫q(r)dr. In the case, the dipole moments are acquired by the DFT or an experiment, for example.

Furthermore, not only the charge information but the total energy of the plurality of electrons may be used as the teacher data. In the case, the learning for the GNN before learning is executed in the procedure of the processing in FIG. 5 by propagating back an error between the total energy based on the result obtained by inputting the training data to the GNN before learning and the total energy of the teacher data along a procedure opposite to the procedure of the processing in the flowchart of FIG. 5.

For example, the learning unit inputs the graph including the plurality of atoms represented by the feature amounts to the GNN before learning as training data, and determines the plurality of charges corresponding to the plurality of atoms by the charge balance method using the plurality of parameters corresponding to the plurality of atoms output from the GNN. Next, the learning unit may learn the GNN by updating a plurality of weights in the GNN. The update may be performed by converting the difference between the plurality of charges in the correct answer data corresponding to the training data and the plurality of determined charges into a plurality of parameters via an inverse operation of the charge balance method and propagating back the plurality of parameters obtained by the conversion in the GNN before learning.

The learning apparatus according to the embodiment can learn the GNN used in the estimation processing in FIG. 5. The GNN 311 learned by the learning apparatus can achieve the effects in the embodiment by being combined with the charge balance method performed by the charge determination unit 313 and being combined with the energy determination unit 315.

When the technical idea in the embodiment is achieved by an estimation method, the estimation method inputs a feature amount of each of a plurality of atoms to a neural network to update the feature amount by at least one processor, a generates a parameter corresponding to each of the plurality of atoms based on the updated feature amount by at least one processor, and determines each of a plurality of charges corresponding to each of the plurality of atoms by using the parameter by at least one processor. For example, the estimation method may represent a plurality of atoms in feature amounts, and input the structure of a substance including the plurality of atoms as a graph. The estimation method may calculate the updated feature amounts by maintaining the graph and repeating graph convolution of the feature amounts within a preset cutoff range. The estimation method may output a plurality of parameters corresponding to the plurality of atoms based on the calculated feature amounts. The estimation method thus may determine a plurality of charges corresponding to the plurality of atoms by the charge balance method using the plurality of parameters. Since the procedure and effects of the estimation processing related to the estimation method are similar to those described in the embodiment, the description thereof will be omitted.

When the technical idea in the embodiment is achieved by an estimation program, the estimation program may cause a computer to represent a plurality of atoms in feature amounts, and input the structure of a substance including the plurality of atoms as a graph. The estimation program may cause the computer to calculate the updated feature amounts by maintaining the graph and repeating graph convolution of the feature amounts within a preset cutoff range. The estimation program may cause the computer to output a plurality of parameters corresponding to the plurality of atoms based on the calculated feature amounts. The estimation program thus may cause the computer to determine a plurality of charges corresponding to the plurality of atoms by the charge balance method using the plurality of parameters.

For example, estimation image generation processing can be performed by installing the estimation program in a computer of various analysis apparatuses and analysis servers that analyze energy and/or force related to the plurality of atoms and developing the estimation program on a memory. In the case, a program capable of causing a computer to execute the approach can be distributed by being stored in a storage medium such as a magnetic disk (e.g., hard disk), an optical disk (e.g., CD-ROM and DVD), and a semiconductor memory. Since the procedure and effects of the estimation processing related to the estimation program are similar to those described in the embodiment, the description thereof will be omitted.

A part or all of each apparatus in the above-described embodiment may be configured by hardware, or may be configured by information processing of software (program) executed by a CPU, a GPU, or the like. When configured by information processing by software, the information processing of software may be executed by storing software that implements at least a partial function of each apparatus in the above-described embodiment in a non-transitory storage medium (non-transitory computer readable medium) such as a flexible disk, a compact disc-read only memory (CD-ROM), and a USB memory and causing the computer 30 to read the software. Furthermore, the software may be downloaded via the communication network 5. Moreover, the information processing may be executed by hardware by implementing software in circuitry such as an ASIC and an FPGA.

The type of a storage medium that stores the software is not limited. The storage medium is not limited to a removable storage medium such as a magnetic disk and an optical disk, and may be a fixed storage medium such as a hard disk and a memory. Furthermore, the storage medium may be provided inside the computer, or may be provided outside the computer.

In the present specification (including claims), an expression of “at least one of a, b, and c” or “at least one of a, b, or c” (including similar expressions) includes any of a, b, c, a-b, a-c, b-c, and a-b-c. Furthermore, a plurality of instances may be included for any element, such as a-a, a-b-b, and a-a-b-b-c-c. Moreover, an element other than listed elements (a, b, and c), such as d of a-b-c-d, may be added.

In the present specification (including claims), cases where expressions such as “data as input/based on/in accordance with/in response to data” (including similar expressions) are used include a case where various pieces of data itself are used as input and a case where various pieces of data subjected to some kind of processing (e.g., noise added data, normalized data, and intermediate expressions of various pieces of data) are used as input, unless otherwise specified. Furthermore, when it is described that some kind of result is obtained “based on/in accordance with/in response to data”, a case where the result is obtained based only on the data is included, and a case where the result is obtained under the influence of other data, factors, conditions, and/or states other than the data may also be included. Furthermore, when it is described that “data is output”, a case where various pieces of data itself are used as output and a case where various pieces of data subjected to some kind of processing (e.g., noise added data, normalized data, and intermediate expressions of various pieces of data) are used as output are included, unless otherwise specified.

In the present specification (including claims), the terms “connected” and “coupled” are intended to be non-limiting terms including all of direct connection/coupling, indirect connection/coupling, electrical connection/coupling, communicative connection/coupling, operative connection/coupling, physical connection/coupling, and the like. The terms should be appropriately interpreted in accordance with the context in which the terms are used. Connection/coupling forms which are not intentionally or naturally excluded should be interpreted in a non-limiting manner as being included in the terms.

In the present specification (including claims), an expression “A configured to B” may include that the physical structure of an element A has a configuration capable of executing an operation B, and that a permanent or temporary setting/configuration of the element A is configured/set to actually execute the operation B. For example, when the element A is a general-purpose processor, the processor may have a hardware configuration capable of executing the operation B, and the processor is only required to be configured to actually execute the operation B by setting of the permanent or temporary program (command) setting. Furthermore, when the element A is a dedicated processor, dedicated arithmetic circuitry, and the like, the circuitry structure of the processor is only required to be implemented to actually execute the operation B regardless of whether or not a control command and data are actually attached.

In the present specification (including claims), terms meaning inclusion or possession (e.g., “comprising/including” and “having”) are intended as open-ended terms including a case where objects other than targets indicated by objects of the terms are included or possessed. When an object of a term meaning inclusion or possession is an expression that does not designate number and quantity or an expression that suggests a singular number (expression with article of “a” or “an”), the expression should be interpreted as not being limited to a certain number.

In the present specification (including claims), even if an expression such as “one or more” or “at least one” is used in a part and an expression that does not designate number and quantity or an expression that suggests a singular number (expression with article of “a” or “an”) is used in another part, the latter expression is not intended to mean “one”. In general, the expression that does not designate number and quantity or the expression that suggests a singular number (expression with article of “a” or “an”) should be interpreted as not necessarily being limited to a certain number.

In the present specification, when it is described that a certain effect (advantage/result) is obtained in a certain configuration of a certain example, it should be understood that the effect is obtained in one or a plurality of other examples having the configuration unless there is some special reason. Note, however, that it should be understood that the presence or absence of the effect generally depends on various factors, conditions, and/or states, and that the effect is not necessarily obtained by the configuration. The effect is merely obtained by the configuration described in an example when various factors, conditions, and/or states are satisfied. The effect is not necessarily obtained in the invention according to claims in which the configuration or a similar configuration is specified.

In the present specification (including claims), terms such as “maximize” include determining a global maximum, determining an approximation of the global maximum, determining a local maximum, and determining an approximation of the local maximum, and should be appropriately interpreted depending on the context in which the terms are used. Furthermore, stochastically or heuristically determining an approximation of the maximum is included. Similarly, terms such as “minimize” include determining a global minimum, determining an approximation of the global minimum, determining a local minimum, and determining an approximation of the local minimum, and should be appropriately interpreted depending on the context in which the terms are used. Furthermore, stochastically or heuristically determining an approximation of the minimum is included. Similarly, terms such as “optimize” include determining a global optimum, determining an approximation of the global optimum, determining a local optimum, and determining an approximation of the local optimum, and should be appropriately interpreted depending on the context in which the terms are used. Furthermore, stochastically or heuristically determining an approximation of the optimum is included.

In the present specification (including claims), when a plurality of pieces of hardware performs predetermined processing, the pieces of hardware may perform the predetermined processing in cooperation with each other, or some pieces of hardware may perform all of the predetermined processing. Furthermore, some hardware may perform a part of the predetermined processing, and other hardware may perform the rest of the predetermined processing. In the present specification (including claims), when expressions such as “one or a plurality of pieces of hardware performs first processing, and the one or the plurality of pieces of hardware performs second processing” are used, the hardware that performs the first processing and the hardware that performs the second processing may be the same as or different from each other. That is, the hardware that performs the first processing and the hardware that performs the second processing are required to be included in the one or the plurality of pieces of hardware. Note that hardware may include electronic circuitry or an apparatus including electronic circuitry.

In the present specification (including claims), when a plurality of storage devices (memories) stores data, each of the plurality of storage devices (memories) may store only a part of the data, or may store the entire data.

What is claimed is:

1. An estimation apparatus comprising:
at least one memory; and
at least one processor,
wherein the at least one processor inputs a feature amount of each of a plurality of atoms to a neural network to update the feature amount, and generates a parameter corresponding to each of the plurality of atoms based on the updated feature amount, and
determines each of a plurality of charges corresponding to each of the plurality of atoms by using the parameter.

2. The estimation apparatus according to claim 1,
wherein each of a plurality of pieces of energy corresponding to each of the plurality of atoms is determined based on the plurality of charges and the feature amount that has been calculated.

3. The estimation apparatus according to claim 2,
wherein the plurality of pieces of energy is determined by a neural network.

4. The estimation apparatus according to claim 2,
wherein charge balance energy of the plurality of atoms is determined based on the plurality of charges and the parameter.

5. The estimation apparatus according to claim 4,
wherein total energy over the plurality of atoms is determined based on the plurality of pieces of energy and the charge balance energy.

6. The estimation apparatus according to claim 1,
wherein the parameter includes, in each of the plurality of atoms, at least one of an index indicating a degree of easiness of being electrically biased and an index indicating a degree of hardness of charge variation.

7. The estimation apparatus according to claim 1,
wherein the neural network is a graph neural network.

8. The estimation apparatus according to claim 7,
wherein the feature amount that has been updated is calculated by maintaining a graph indicating a structure of a substance including the plurality of atoms and repeating graph convolution of the feature amount within a preset cutoff range.

9. The estimation apparatus according to claim 1,
wherein the plurality of charges corresponding to the plurality of atoms is determined by a charge balance method using the parameter.

10. The estimation apparatus according to claim 1,
wherein the parameter is output as a common parameter for the same elements in the plurality of atoms.

11. The estimation apparatus according to claim 1,
wherein the feature amount of each of the plurality of atoms is expressed as a matrix.

12. The estimation apparatus according to claim 1,
wherein the parameter is generated by performing linear transformation on the updated feature amount.

13. An estimation method comprising:
inputting, by at least one processor, a feature amount of each of a plurality of atoms to a neural network to update the feature amount,
generating, by the at least one processor, a parameter corresponding to each of the plurality of atoms based on the updated feature amount, and
determining, by the at least one processor, each of a plurality of charges corresponding to each of the plurality of atoms by using the parameter.

14. The estimation method according to claim 13, further comprising:
determining, by the at least one processor, each of a plurality of pieces of energy corresponding to each of the plurality of atoms based on the plurality of charges and the feature amount that has been calculated.

15. The estimation method according to claim 14,
wherein the plurality of pieces of energy is determined by a neural network.

16. The estimation method according to claim 14, further comprising:
determining, by the at least one processor, charge balance energy of the plurality of atoms based on the plurality of charges and the parameter.

17. The estimation method according to claim 16, further comprising:
determining, by the at least one processor, total energy over the plurality of atoms based on the plurality of pieces of energy and the charge balance energy.

18. The estimation method according to claim 13,
wherein the parameter includes, in each of the plurality of atoms, at least one of an index indicating a degree of easiness of being electrically biased and an index indicating a degree of hardness of charge variation.

19. The estimation method according to claim 13,
wherein the neural network is a graph neural network.

20. The estimation method according to claim 19,
wherein the feature amount that has been updated is calculated by maintaining a graph indicating a structure of a substance including the plurality of atoms and repeating graph convolution of the feature amount within a preset cutoff range.
